(19) United States
(12) Patent Application Publication
(10) Pub. No.: US 2023/0255836 A1
(43) Pub. Date: Aug. 17, 2023

(54) ABSORBENT ARTICLES

(71) Applicant: Joii Ltd, Dublin (IE)

(72) Inventors: Christopher VERNALL, Dublin (IE); Andrew VERNALL, London (GB)

(21) Appl. No.: 18/303,867

(22) Filed: Apr. 20, 2023

Related U.S. Application Data
(63) Continuation of application No. PCT/EP2021/079629, filed on Oct. 26, 2021.

(30) Foreign Application Priority Data
Oct. 27, 2020 (GB) 2017002.3

(51) Int. Cl. A61F 13/537; A61F 13/472

(52) U.S. Cl. CPC A61F 13/53747; A61F 13/472; A61F 13/53717; A61F 2013/53445

(57) ABSTRACT

An absorbent article comprising a body-facing layer; a liquid-impermeable garment-facing layer; and an absorbent structure therebetween; wherein the absorbent article further comprises a wicking layer disposed between the body-facing layer and the absorbent structure; and an acquisition/dispersion (ADL) layer disposed on the opposite side of the absorbent structure from the garment-facing layer; wherein the body-facing layer comprises at least one barrier sheet, wherein the barrier sheet is substantially liquid-impermeable and is discontinuous, to allow the passage of liquid therethrough.

### CROSS-REFERENCE TO RELATED APPLICATIONS

This application is a continuation under 35 U.S.C. § 120 of International Application No. PCT/EP2021/079629, filed Oct. 26, 2021 which claims priority to United Kingdom Application No. GB 2017002.3, filed Oct. 27, 2020, under 35 U.S.C. § 119(a). Each of the above-referenced patent applications is incorporated by reference in its entirety.

### BACKGROUND

### Field of the Invention

The present invention relates to absorbent articles comprising a wicking layer and a barrier sheet, which together facilitate improved efficiency in distributing and storing liquids. The absorbent articles are disposable absorbent articles, such as sanitary napkins, panty-liners or incontinence pads.

### Description of the Related Technology

Absorbent articles such as sanitary towels/napkins/pads and incontinence pads are designed to rapidly absorb and contain liquid which is incident on a portion of the article adjacent to an orifice; most often a central portion of the article. Presently available articles generally fail to make efficient use of their entire storage capacity, which is a result of inefficient transport of the liquid from the point of ingress towards peripheral zones of the article.

Presently available articles comprise a liquid permeable body-facing layer and a liquid impermeable garment-facing layer, with an absorbent structure therebetween. In some cases, sanitary napkins and incontinence pads further comprise an acquisition-distribution layer (ADL), which functions to rapidly draw liquid away from a point of ingress and convey the liquid towards the absorbent structure. Primarily the purpose of the ADL is to provide a temporary reservoir for the liquid and facilitate movement of the liquid into the absorbent structure, and the ADL is poor at conveying the liquid to peripheral regions of the article. When wearers of such articles experience heavy flow, the absorbent structure beneath and immediately adjacent to the point of liquid ingress can become quickly saturated. Furthermore, the ADL and absorbent structure proximal to the liquid ingress region collects and stores the majority of the liquid insult, whilst absorbent structures situated at distal regions of the article are underutilised, increasing the risk of leaks from the central portion of the article. During times of heavy flow, when liquid does eventually reach distal regions of the article, there are often problems with liquid escaping from the body-facing layer of the article back to the skin of the user, so called “rewetting”. Rewetting becomes a particular concern when wicking towards peripheral regions is enhanced, because liquid is spread over a wider surface area and so may transfer to the wearer's skin at areas distant from the liquid ingress region.

Consequently, there is a need for absorbent articles with a storage capacity which can be fully and efficiently utilised, whilst minimising rewetting at peripheral regions.

### SUMMARY

The present invention provides an absorbent article comprising a body-facing layer; a liquid-impermeable garment-facing layer; and an absorbent structure therebetween; wherein the absorbent article further comprises a wicking layer disposed between the body-facing layer and the absorbent structure; and an acquisition/dispersion (ADL) layer disposed on the opposite side of the absorbent structure from the garment-facing layer; wherein the body-facing layer comprises at least one barrier sheet, wherein the barrier sheet is substantially liquid-impermeable and is discontinuous, to allow the passage of liquid therethrough.

Further features and advantages of the invention will become apparent from the following description of preferred embodiments of the invention, given by way of example only, which is made with reference to the accompanying drawings.

### BRIEF DESCRIPTION OF THE DRAWINGS

FIG. 1 shows an exemplary absorbent article of the invention.

FIG. 2 shows exemplary absorbent articles of the invention, with a representation of how liquid moves through the articles when a wicking layer is incorporated.

FIG. 3 shows how the length of the absorbent article may be increased.

FIG. 4 shows an exemplary apparatus for performing a wicking rate (capillarity) experiment as described elsewhere herein.

FIG. 5 shows a graph containing the results of liquid wicking rate tests, which are described in example 3 herein.

FIG. 6 shows a graph containing further results of liquid wicking rate tests, which are described in example 3 herein.

FIG. 7 shows a graph containing the results of liquid absorptive capacity tests, which are described in example 4 herein.

FIG. 8 shows a graph containing further results of liquid absorptive capacity tests, which are described in example 4 herein.

FIG. 9 shows a graph containing the results of liquid spreading area tests, which are described in example 5 herein.

FIG. 10 also shows photographs and diagrammatic representations of liquid spreading area tests, which are described in example 5 herein.

FIG. 11 shows how certain placements of the barrier layer have comparable effects on liquid spreading behaviour, as discussed in example 6 herein.

### DETAILED DESCRIPTION

Absorbent articles of the invention are planar in shape, comprising a longitudinal axis extending from two mutually opposed end portions. In some embodiments, a first end portion of the article comprises a front portion, which during use is orientated towards the ventral side of the user; a second end portion of the article comprises a rear portion, which during use is orientated towards the dorsal side of the user. Absorbent articles of the invention may be unidirectional, i.e. intended to be worn with a front portion orientated towards a ventral side of the user, or bi-directional, i.e. wherein the front and rear portions are substantially the same and the article may be worn with either end portion orientated towards the ventral side of the user.

The body-facing layer comprises an upper surface area, which in use is orientated towards the body of the wearer. In use, the liquid insult is incident upon the body-facing layer.

The article comprises three portions; a central portion, and two mutually opposed end portions either side of the central portion. When the absorbent article is worn by a user the end portions are orientated dorsally and ventrally. The point of liquid ingress is substantially within the central portion of the upper surface area.

The absorbent article of the invention may be a sanitary towel/napkin/pad or an incontinence pad.

In some embodiments the absorbent article is a sanitary napkin or an incontinence pad which is longer than an average sanitary napkin or incontinence pad. Absorbent articles (e.g. sanitary napkins) with increased length are particularly suitable for use by women experiencing heavy menstrual flow, or for night-time wear. In some embodiments the absorbent article has a length equal to or greater than about 170 mm; for example, greater than about 180 mm, greater than about 190, or greater than about 200 mm. In some embodiments the absorbent article has a length of 150-300 mm; suitably from 200-300 mm. When the absorbent article is increased in length this of course increases the storage capacity of the article. However, in an ordinary absorbent article liquid may not be efficiently transported from a point of ingress to make use of the increased length. This problem is solved by the absorbent article of the invention, since the wicking layer moves liquid away from the point of ingress to end portions of the article and then the barrier sheet prevents rewetting at the end portions.

### Wicking Layer

Absorbent articles of the invention comprise a wicking layer. Suitably, the wicking layer is arranged to facilitate lateral movement of liquid from a region of liquid ingress (e.g. a central region of a body-facing layer) towards at least one end/distal portion of the article. In some embodiments the wicking layer is arranged to facilitate movement of liquid from a central region of the article to peripheral regions of the article, such as the end portions (e.g. the front and back portions) of the article, which are positioned at opposite ends along the longitudinal axis of the article. The absorbent article comprises a wicking layer in addition to an ADL. Optionally the wicking layer is disposed between the ADL and an absorbent structure. The ADL facilitates rapid transfer of liquid away from the skin and the wicking layer facilitates lateral movement of the liquid towards peripheral portions (e.g. the end portions) of the article prior to its transfer to the absorbent structure for storage. Consequently, including a wicking layer in absorbent articles of the invention serves to maximise utilisation of the storage capacity of the absorbent article. Inclusion of the wicking layer means that the absorbent structure may be used more efficiently to store menstrual fluid. This could allow the thickness of the absorbent structure to be reduced whilst its storage capacity remains high, resulting in a thinner pad which remains absorbent, whilst being more comfortable for the wearer.

In some embodiments, the material used in the wicking layer has a high liquid wicking rate, which facilitates movement toward outer edges (e.g. the end portions) of the absorbent article. The wicking rate of a material may be defined in terms of its “Vertical Wicking Rate” which refers to the ability of a material to draw fluids in a vertical direction against gravity. Vertical wicking is a desirable property since it indicates a material's ability to move fluid away from a point of contact. One measure of a given material's vertical wicking performance is the Vertical Wicking Height—i.e., the distance that the material can wick liquid in a direction opposite to gravitational force. In some embodiments of the invention, the wicking layer comprises a material which exhibits a Vertical Wicking Height of at least 100 mm within 3 minutes when measured using EDANA NWSP 101.1 RO (20). In some embodiments, the wicking layer absorbs and wicks a sufficient amount of liquid per gram of the material so as to have a liquid flux adequate to substantially move the liquid out of the insult area prior to the next liquid insult.

Test for Liquid Wicking Rate (Capillarity)

Fabrics having suitable liquid wicking rates may be identified in experiments performed in accordance with Edana Nonwovens Standard Procedure 010.1.RO (20). According to the standard test, ‘liquid wicking rate’ is a measure of the capillarity of the test material which is the rate at which a liquid is transported into the fabric by capillary action. The capillarity method measures the rate of vertical capillary rise in a specimen strip suspended in a test liquid. In some embodiments, the wicking layer comprises a material having a liquid wicking rate of at least 80 mm, optionally at least 100 m, within 3 minutes when measured using EDANA NWSP 101.1 RO (20). The procedure for determining liquid wicking rate in accordance with this standard is as follows:
1. Cut at least 5 test specimens (30±1) mm wide×(250±1) mm long in both the machine direction and the cross direction.
2. Punch two holes, (5±1) mm diameter out of one of the short ends of each test specimen at (5±1) mm from short and long sides.
3. The liquid shall be left long enough to equilibrate with the conditioned atmosphere.
4. Clamp the test specimen vertically to the horizontal support with the punched holes at the bottom.
5. Place a glass rod through two slots to keep tension on the test specimen and maintain it vertically.
6. Place test specimen neat and parallel to the measuring rod and projecting (15±2) mm below the zero point of the measuring rod.
7. Lower the horizontal support until the zero point of the measuring rod touches the liquid surface (the lower test specimen edge is then (15±2) mm below the surface.
8. At this moment, start stopwatch.
9. Record the height of capillary rise of the liquid after 10 s, 30 s, 60 s (and 300 s if required). (If capillary rise is not a uniform straight line, record the highest point)
10. Repeat 4 to 9 with the other 9 test specimens.
11. Use fresh conditioned test liquid for each set of 10 test specimens.
12. Calculate the average capillarity rise obtained on the 5 test specimens for each specified time and the standard deviation:
   a) in the machine direction
   b) in the cross direction
13. Trace a curve using the data obtained (12), so that the wicking rate can be calculated at a required time or at a required capillary rise.

Liquid Absorptive Capacity

In some embodiments the wicking layer comprises a material which has high liquid absorptive capacity when measured using EDANA NWSP 101.1 RO (20). According to the standard test, ‘liquid absorptive capacity’ is the mass of liquid that is absorbed by unit mass of the test material expressed as a percentage of the mass of the test material under specified conditions and after a specified time. The absorptive capacity method performed according to the above standard provides a measure of the amount of liquid held within a test specimen after specified times of immersion and drainage. This method measures the liquid stored within the test specimen itself after drainage has occurred vertically. Liquid absorptive capacity may be calculated by measuring the mass of a material after it has been submerged in 0.9% saline solution for 60 seconds and expressing the result as a percentage of the original weight of the material prior to wetting. In some embodiments, the wicking layer comprises a material having a liquid absorptive capacity of at least 600% when measured using EDANA NWSP 101.1 RO (20). The procedure for determining liquid absorptive capacity in accordance with this standard is as follows:
1. Cut 5 test specimen (100±1) mm×(100±1) mm from the sheet. If the individual test specimen weighs less than 1 g, lay test specimens on top of each other to give to each of the piles a total stacked mass of at least 1 g.
2. The liquid shall be left long enough to equilibrate with the conditioned atmosphere.
3. Weigh the test specimen (or pile) to an accuracy of 0.01 g, using the balance and the weighing glass with cover.
4. Place the test specimen (or pile) on the stainless steel gauze, fastening it (them) at the edges with the clips.
5. Place the gauze with the attached test specimen(s) approximately 20 mm below the liquid surface in the dish and start the stopwatch. Introduce the gauze obliquely in order to avoid trapping air bubbles.
6. After (60±1) s remove the gauze test specimen support and test specimen (or pile).
7. Remove all clips but one at one corner.
8. Hang freely vertical to drain for (120±3) s.
9. Take the test specimen (or pile) off the gauze without squeezing the liquid from it; place the test specimen in the weighing glass with cover and weight.
10. Repeat 3 to 9 for the other 4 test specimens.
11. Use fresh conditioned test liquid for each set of 5 test specimens (or piles).
12. Calculate:
   a) The liquid absorptive capacity (LAC) in % of each specimen or each pile from the following:

Mn−Mk

LAC %= - - - ×100%
Wherein ‘Mk’ is mass in grams of the dry test specimen(s)
Mn is mass in grams of the wet test specimen(s) at the end of the test.
b) The average liquid absorptive capacity of the 5 test specimens (or piles) and the standard deviation.

In some embodiments the wicking layer comprises parallel laid fibres. It has been found by the inventors that materials comprising fibres which are parallel laid (meaning the fibres are mostly aligned in the machine direction) are particularly suitable for use in the wicking layer. Whilst not wishing to be bound by theory, it is believed that when the direction of the fibres is aligned with the longitudinal axis of the article, wicking towards the end portions of the article, i.e. those positioned at either end along the longitudinal axis of the article, is facilitated by capillary forces.

The inventors have also found that when the wicking layer comprises a material comprising apertures, wicking can be improved. Without wishing to be bound by theory, it is believed that introducing apertures into the material compacts adjacent fibres together, decreasing which decreases space between the fibres, and so increases capillary forces and so improves wicking of liquid along bundles of the fibres. In some embodiments the wicking layer comprises a sheet of fibrous material comprising 10-40 apertures per in2 (or 1-7 apertures per cm2); for example, 15-30, or 20-25 apertures per in2 (or 2-5, or 3-4 apertures per cm2). In an exemplary embodiment, the wicking layer comprises a sheet of fibrous material comprising 22 apertures per in2 (or about 3 apertures per cm2). Suitable materials for use in the wicking layer may contain apertures of various sizes. The fibrous material comprising the apertures may alternatively be referred to as a mesh; for example, ‘22 mesh’ means there are 22 apertures per in2.

Nonwoven fabrics suitable for use in the wicking layer may include drylaid, wetlaid or spunlaid formations or a composite structure of the aforementioned methods. Some potential structures may include: carded and parallel laid hydroentangled (spunlace) fabrics (with or without apertures and with or without channels or cavities); wetlaid fabric with orientation in a machine direction, hydroentangled, thermally or chemically bonded; carded and parallel laid and thermally bonded structures bonded by calendaring or through-air methods; carded parallel laid and chemically bonded structures.

In some embodiments, the wicking layer comprises a sheet of material comprising woven or non-woven fibres. In some embodiments the fibre orientation of the non-woven fibres is in a single direction or in one or more directions. Examples of suitable non-woven fibres include “spunlaces”. Spunlace is a non-woven fabric, formed by using waterjets to bond a carded web of fibres. To form a spunlace, water is emitted under high pressure and velocity from closely positioned nozzles onto a web of loose fibres. The intensity of the water stream and the pattern of the supporting drum or belt entangle, spin and curl the fibres about one another. The entangling of the fibres and the friction between these yields a cohesive web. The process makes a nonwoven fabric with physical properties of softness, high bulk, drapability, stretchiness, good strength and depending upon the fibre used, aesthetics that mimic traditional knitted or woven textiles. When the wicking layer comprises a non-woven mat of fibres, wicking along the material is improved. Without wishing to be bound by theory, it is thought that when a non-woven fibre is used, capillary forces along the fibres are not regularly interrupted as they may be in fabrics or materials which are woven in a regular pattern.

In some embodiments the wicking layer comprises biodegradable or compostable fibres. In some embodiments, the wicking layer comprises bamboo or viscose fibres. In some embodiments the wicking layer comprises natural fibres such as cotton and bamboo which have been scoured and de-waxed to enhance their hydrophilicity.

In some embodiments the wicking layer comprises synthetic fibres comprising a hydrophilic surface. Base polymers for the synthetic fibres may include polyesters, polyamides, polyacrylates or polyolefins. In some embodiments the fibres comprise a hydrophilic surface which is applied using at least one of hydrophilic surfactants; hydrophilic plasma treatment; hydrophilic additives; hydrophilic binders and/or hydrophilic coatings. In some embodiments the wicking layer comprises man-made fibres that are inherently wettable such as fibres from regenerated cellulose including polynosics, modal, viscose, cuprammonium rayon, lyocell, veocel, and regenerated protein fibres such as silk. The fibres may comprise synthetic or regenerated fibres which comprisee non-circular fibre cross-sections to increase their absorbency, these may include: trilobal fibres, 4DG fibres (i.e. fibres comprising thermoplastic polymer, e.g. PET, PP or Nylon, which contain 6-10 grooves or channels along the longitudinal axis of the fibre), hexaflower fibres, ribbon fibres; triangular fibres and hollow fibres. In some embodiments the wicking layer comprises microfibres, which promote wicking by forming structures with small capillaries increasing the capillary forces driving the liquid flow. Microfibres are fibres with a diameter below 10 μm and are either extruded and drawn, formed by splitting of segmented pie bicomponent fibres of higher diameter into segments with microfibre diameter range, or ‘islands-in-the-sea’ bicomponent fibres where the matrix (sea) is dissolved and the microfibres (islands) form the structure.

In some embodiments the wicking layer comprises a sheet of fibrous material having a fabric density of 20-60 g/m2; for example, 30-50 g/m2. For example, a sheet of non-woven (e.g. spunlace) fibres having a fabric density of 20-60 g/m2.

In an exemplary embodiment, the wicking layer comprises a sheet of parallel-laid spunlace cellulosic fibres, wherein the sheet has a fabric density of 20-60 g/m2 and comprises 15-30 apertures per in2 (i.e. ‘15-30 mesh’ or 2-5 apertures per cm2). In a further example, the wicking layer comprises a sheet of parallel-laid spunlace cellulosic fibres, wherein the sheet has a fabric density of 25-55 g/m2 and comprises 20-25 apertures per in2 (i.e. ‘20-25 mesh’ or 3-4 apertures per cm2). For example, the wicking layer may comprise a sheet of parallel-laid spunlace bamboo or viscose fibres, wherein the sheet has a fabric density of 25-55 g/m2 and comprises 20-25 apertures per in2 (i.e. ‘20-25 mesh’ or 3-4 apertures per cm2).

### Barrier Sheet

The absorbent article of the invention comprises a wicking layer; therefore, liquid may be transported to and stored over a wider area of the absorbent structure including the absorbent structure at the end portions of the article. This is advantageous, because the full storage capacity of the article may be used efficiently. However, this could also result in rewetting from areas of the body-facing layer distant from the region of liquid ingress, such as at the end portions of the article. When the body-facing layer of the absorbent article comprises a discontinuous liquid impermeable barrier sheet, rewetting from the body-facing layer may be mitigated. The barrier sheet and wicking layer act synergistically, in that the wicking layer facilitates movement of liquid from the point of ingress, towards the end portions of the article, whilst the barrier layer prevents rewetting occurring over the resulting increased storage area. Consequently, use of a wicking layer and barrier sheet allows efficient utilisation of the full storage capacity of the article whilst minimising leakage and rewetting via the body-facing layer.

Absorbent articles of the invention comprise a body-facing layer comprising a barrier sheet, wherein the barrier sheet is substantially liquid-impermeable/impervious and is discontinuous to allow the passage of liquid therethrough. In some embodiments, the barrier sheet is arranged to prevent rewetting from the body-facing layer onto the skin of the wearer, whilst allowing ingress of liquid at a region where the liquid is incident upon the article.

In some embodiments the end portions (e.g. a front portion and a rear portion) of the body-facing layer comprise the barrier sheet and a central portion of the body-facing layer is substantially free from the barrier sheet. In other words, the article comprises end portions at opposite ends along the longitudinal axis of the article, which comprise a body-facing layer comprising the barrier sheet. In some embodiments the barrier sheet is continuous over a front portion and a rear portion of the body-facing layer; in cases were the article is bi-directional (i.e. may be worn with either end orientated to the front of a wearer's body), the barrier sheet is continuous over the end portions at opposite ends along the longitudinal axis of the article.

In some embodiments, at least 20% of the surface area of the body-facing layer comprises the barrier sheet. For example, 30%-70% of the body-facing layer comprises the barrier sheet. In some such embodiments, the barrier sheet is equally proportioned between the end portions of the body-facing layer; that is, the end portions at opposite ends along the longitudinal axis of the absorbent article.

The barrier sheet of the article is substantially liquid impermeable, which serves to reduce rewetting. In some embodiments, the barrier sheet is arranged to reduce rewetting from the end portions of the article. The barrier sheet is ideally discontinuous, so that a liquid insult onto the body-facing layer can pass the barrier layer to be received by the ADL and wicking layer. In some embodiments the body-facing layer at the region of liquid ingress is free from barrier sheet. Alternatively, the barrier sheet may comprise openings or apertures in and around the region of liquid ingress. For example, the barrier sheet may comprise regular apertures over a central portion of the body-facing layer. In some embodiments the body-facing layer comprises the barrier sheet and ADL, wherein the ADL is continuous with the barrier sheet. In some such embodiments the region of liquid ingress at the central region of the body facing layer comprises the ADL, whilst the end portions of the body-facing layer comprise the barrier sheet. In other embodiments the ADL is provided between the barrier sheet and the wicking layer.

In some embodiments the barrier sheet comprises a plastic film; such films may comprise polyolefins, such as polyethylene, polypropylene or bio polyethylene, polyesters, including recycled, bio-based and biodegradable types, polyvinyl alcohol, polyvinyl chloride, and ethylene vinyl alcohol; thermoplastic elastomers including thermoplastic olefins, thermoplastic polyurethanes, thermoplastic polyamides, thermoplastic styrenic compounds, thermoplastic vulcanised compounds, and thermoplastic co-polyester compounds; regenerated cellulose and cellulose acetate; natural and synthetic rubbers, and any mixtures of the aforementioned polymers. Other materials which may be incorporated into the barrier sheet include a woven, knitted, or nonwoven fabric coated with a hydrophobic and barrier coating: coatings can include acrylic and their blends such as styrene acrylate, polyvinyl alcohols, polyvinyl chlorides, latex binders, polyurethane binders and fluoropolymers. The barrier sheet may alternatively comprise a microporous membrane, or a high density woven, knitted or nonwoven non-absorbent structure which act as a barrier to liquid.

Other materials/structures which the barrier material may comprise include liquid impermeable nonwoven fabric such as a flat roller calendared, heat pressed or flatbed laminated structures; a nanoporous electrospun layer designed as a barrier layer; a laminated structure containing a woven, knitted or nonwoven and/or film; nonwoven, woven, knitted or other fabric with a hydrophobic finish (hydrophobic finishes may include those based on waxes including synthetic or natural waxes, fluorocarbons, and silicone based materials); fluoropolymer based fibres such as polytetrafluoroethylene constructed into a fabric; fibres in a nonwoven structure with surface modification treatment to increase their hydrophobicity, such as plasma treatment or chemical treatment.

The barrier sheet should be thin enough to be flexible, so as to conform with the shape of the wearer but be thick enough to avoid tearing during use. In some embodiments the thickness of the barrier sheet is 10-50 μm; for example, 15-30 μm or 20-25 μm.

In alternative embodiments, the barrier sheet is substantially liquid impermeable in one direction, but is permeable to liquid in the opposite direction; for example, the barrier sheet may be dual-sided to allow passage of liquid towards the ADL and wicking layer, but prevent back travel of liquid in the opposite direction. For example, an upper (i.e. body-facing) surface and a lower surface of the barrier sheet may be treated differently; for example, the barrier sheet may include a surfactant on the upper surface, so as to facilitate liquid transfer therethrough (especially at a liquid ingress region; e.g., central portion, of the barrier sheet), and include a hydrophobic agent on the lower surface (especially at the end portions of the article) to minimize the liquid contained within the absorbent core from egress through the body-facing layer.

In some embodiments, the body-facing layer comprises a top sheet in addition to a barrier sheet. In some such embodiments, the barrier sheet is disposed between the top sheet and the ADL; however, alternatively, the top-sheet may be disposed between the barrier sheet and the ADL; the barrier sheet may of course also be continuous with the ADL with the topsheet facing an outer surface of the body-facing layer. When the barrier sheet is disposed below the top-sheet the comfort for the wearer may be improved. However, either configuration is suitable for preventing egress of liquid from the storage layer; both designs with the barrier sheet above and below the top-sheet gave no leakage of liquid in the covered area (as discussed in example 6 herein).

Topsheet

In some embodiments the body facing layer comprises a topsheet. Ideally the topsheet fabric within the pad should be wettable (hydrophilic) and non-absorbent. The hydrophilicity enables the fluid to inlet through the structure rather than sit on the surface. The low absorbency means the top-sheet rewet will be low allowing for higher wearer comfort. In some such embodiments the top-sheet comprises synthetic fibre blends including polyethylene, polypropylene and polyester or natural fabrics comprising cotton and viscose. In some embodiments the tops-sheet comprises a non-woven fabric including spunlaid, drylaid or wetlaid structure or a combination of these; nonwoven structures of interest include carded thermally bonded or spunbonded web; spunbond-meltblown-spunbond (SMS) structure or varieties of spunmelt composites (e.g. SMS, SMMS, SMSMMS, etc.). The nonwoven fabric may be bonded by means of thermal bonding such as calendaring, through air bonding, ultrasonic welding, chemical bonding, mechanical bonding such as hydroentanglement, or a combination of bonding mechanisms. The topsheet may also or alternatively comprise perforated plastic films; such as low-density polyethylene, linear low-density polyethylene, ultra-low-density polyethylene, metallocene polyethylene, high-density polyethylene, polypropylene or other polyolefins, polycarbonate, polyvinyl chloride, polyester, polyvinylidene chloride, polyamides, polyurethanes, cellulose acetate, ethylene vinyl acetate, ethylene-vinyl alcohol, natural or synthetic rubbers, or regenerated cellulose cellophane. Alternatively, sustainable bio-based or biodegradable alternatives to these films may be used. In some embodiments the topsheet comprises an embossed film; porous foams; lightweight (<50 g/m2) woven or knitted fabric; or other net/mesh structures that may be woven, knitted or constructed in an alternative method. Fibres that may be used within the topsheet layer can include: natural fibres such as cotton, bast and leaf fibres, protein fibres, bamboo or wood pulp; regenerated fibres such as viscose or lyocell; synthetic fibres including polypropylene, polyethylene, polyester, polyamides, polyacrylates, other polyolefins, bi-component fibres comprising pre-mentioned polymers; blends of natural, regenerated and synthetic fibres; synthetic fibres may contain a hydrophilic surface treatment including surfactants, plasma treatments, additives and coatings or finishes. In some embodiments the topsheet may comprise sustainable (e.g. bio-based or biodegradable) materials, for example the top-sheet may comprise polylactic acid (PLA), polyhydroxyalkanoates (PHA), 3-hydroxybutyrate-co-3-hydroxyvalerate (PHBV), polybutylene succinate (PBS), polyethylene furanoate (PEF), polytrimethylene terephthalate (PTT), thermoplastic starch (TPS), biodegradable Polyethylene terephthalate (PET), biodegradeable polypropylene (PP), polycaprolactone (PCL), polyethylene glycol (PEO), polyglycolic acid (PGA), polyvinyl alcohol (PVA), viscose, hydrophobic viscose, tencell/lyocel, cotton, hydrophobic cotton.

### Backsheet

In some embodiments the liquid impermeable garment facing layer is a backsheet, or comprises a backsheet. In some such embodiments the backsheet comprises a plastic film; for example, polyethylene, polypropylene, ethylene vinyl acetate copolymers, polyurethane, polyisoprene, butadiene-styrene copolymers, styrene block co-polymers, polycarbonate, polyvinyl chloride, polyester, polyvinylidene chloride, polyamides, cellulose acetate, ethylene-vinyl alcohol, natural or synthetic rubbers or regenerated cellulose or sustainable recycled, biobased or biodegradable films. In some embodiments the backsheet comprises a liquid impermeable nonwoven fabric. In some embodiments the backsheet comprises a composite or laminate structure which may contain a nonwoven and/or film structures. In some embodiments the backsheet comprises sustainable recycled, biobased or biodegradable materials such as polylactic acid (PLA), polyhydroxyalkanoates (PHA), polyhydroxybutyrate (PHB), 3-hydroxybutyrate-co-3-hydroxyvalerate (PHBV), polybutylene succinate (PBS), polyethylene furanoate (PEF), polytrimethylene terephthalate (PTT), cellulose acetate, thermoplastic starch (TPS), biodegradeable polyehtylene terephthalate (PET), polyvinyl alcohol (PVA), biodegradeable polypropylene (PP), biodegradeable polyethylene (PE), polycaprolactone (PCL).

### Acquisition-Distribution Layer (ADL)

In some embodiments the ADL comprises nonwoven fabric produced by drylaid, wetlaid or spunmelt methods. These nonwoven fabrics may be bonded through thermal, chemical, or mechanical processes. Nonwoven fabrics which may be used include: carded fabric bonded by through-air bonding, hydroentanglement or needle-punching; airlaid fabrics bonded by spray chemical bonding or thermal bonding (ultrasonic or through-air); meltblown or spunbond fabrics, or a composite of both that may be bonded through calendaring. The nonwoven fabric may further include embossed, creped or textured elements. In some embodiment the ADL comprises woven fabrics including 3D woven fabrics; knitted fabrics including knitted double jersey or spacer fabrics. Fibres that may be used in the above-mentioned fabrics include: natural fibres such as bast or leaf fibres and wood pulp which have been treated or untreated, cotton, treated wool; regenerated fibres such as viscose, bamboo viscose or lyocell; synthetic fibres comprising polypropylene, polyethylene, polyester (PET), polyamides, polyacrylate, polylactic acid (PLA), other polyolefins, bi-component polymers of the pre-mentioned fibres and blends or composites of natural, regenerated, or synthetic fibres. In some embodiments the ADL comprises sustainable (e.g. biodegradeable) materials such as cotton and/or polylactic acid (PLA).

### Absorbent Structure

The absorbent article of the invention comprises an absorbent structure which acts to absorb and store liquid. In some embodiments the absorbent structure comprises an absorbent fibrous material. The fibrous material may comprise fibres with a modified cross-section to increase absorbency which may include but are not limited to splitable fibres, mutilobal fibres, trilobal fibres, 4DG fibres (i.e. fibres comprising thermoplastic polymer, e.g. PET, PP or Nylon, which contain 6-10 grooves or channels along the longitudinal axis of the fibre) or ribbon shaped fibres; microfibres; absorbent nonwoven fabrics produced through drylaid, wetlaid or spunlaid methods with a variety of bonding mechanisms; meltblown fabrics; tissue wraps; airlaid structures; airlaid structures bonded by means of ultrasonic bonding or other thermal or

chemical bonding methods; carded and needlepunched fabrics; absorbent polymeric open-cell foam made of polymers including polyurethane; absorbent sponges; woven or knitted fabrics. Fibres that may be incorporated into the structures include cellulosic wood pulp which is either treated or untreated, cotton fibres, cotton lint or cotton flock, viscose fibres with alternative cross-sections, chemically modified cellulose fibres, plant based fibres with absorbency such as hemp, flax, sodium alginate, calcium alginate, chitin, chitosan. In some embodiments the absorbent structure comprises superabsorbent polymers (SAP) in fibre or particle form; these can include sodium polyacrylate, ionic or non-ionic SAP, potassium-based SAP, citric acid-based SAP, cellulose based SAP; sulfamic acid modified starch (Cargill) based SAP; crosslinked cellulose and protein hydrogels (Tethis); absorbent gelling material; absorbents based on starch or other sustainable alternatives. In some embodiments the absorbent structure comprises sustainable (e.g. biodegradable) SAP materials such as calcium alginate, potato starch, potassium polyacrylate, carboxymethyl cellulose (CMC), chitosan and silica gel.

### SPECIFIC EXAMPLES AND FIGURES

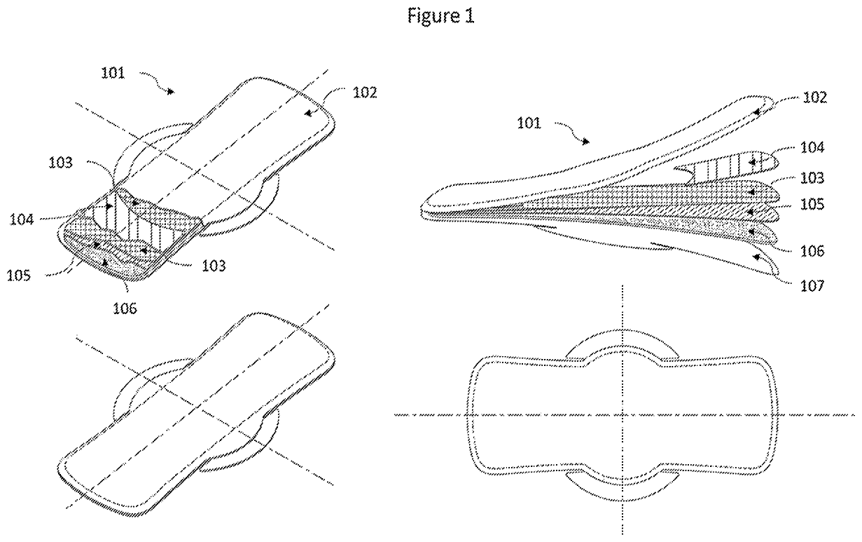

As shown in FIG. 1, an exemplary absorbent article 101 of the invention comprises a body facing layer which comprises a topsheet 102 and a barrier sheet 104. The absorbent article comprises a liquid impermeable garment-facing layer 107 and an absorbent structure 106 (also referred to as an absorbent core) between the body facing layer and the garment-facing layer. Between the body-facing layer and the absorbent structure is a wicking layer 105. An ADL 103 may form part of the body-facing layer, or may be positioned between the barrier sheet 104 and the absorbent structure 106.

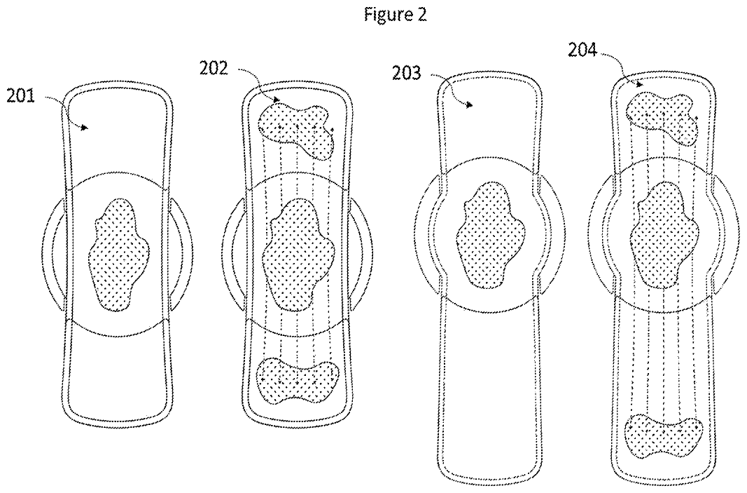

FIG. 2 shows how liquid moves from a point of ingress at the centre of the absorbent article to end portions of the absorbent article when a wicking layer is present. When liquid (e.g. menstrual fluid) is incident on an absorbent article 201, 203, without a wicking layer liquid saturates the central region of the towel closest to the point of ingress. When a wicking layer is present 203 204, liquid is transported towards end portions of the article, maximising the liquid storage capacity of the article and reducing leaks. Also shown in FIG. 2; the inclusion of a wicking layer means that the absorbent article may be increased in length 203 204. Even with increased length the full storage capacity of the article can be used since liquid is transported to end portions of the article through the wicking layer. Inclusion of a barrier sheet prevents rewetting at the end portions of the increased length article.

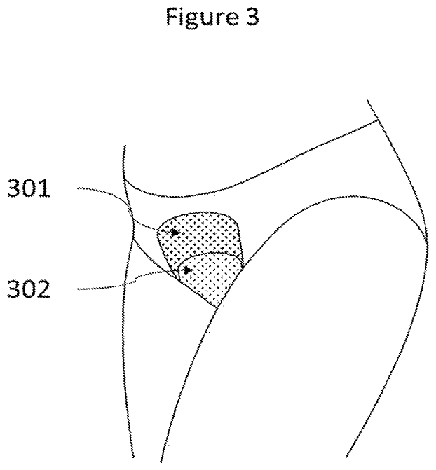

FIG. 3 shows how the storage capacity of an absorbent article of the invention can be further increased by lengthening the article. The article may be increased in length 301 in the forward direction, when compared to a standard-length sanitary towel 302. The enhanced movement of liquid due to incorporation of the wicking layer means that liquid will be moved toward the extended front portion of article. Inclusion of the barrier sheet avoids rewetting from the article at the end portions, reducing rewetting or leakage from an increased length article.

### Example 1

Exemplary materials for use as each sheet component are shown in Table 1 below.

TABLE 1

| Component | Material |
|---|---|
| Topsheet 102 | Hydrophilic Spunbond + meltblown + spunbond SmS |
| | Polypropylene Nonwoven Fabric (8 g/m²) |
| Barrier sheet | Polyethylene film (20 μm thickness) |
| 104 | |
| ADL 103 | Carded air-through bonded - polyester/biocomponent |
| | polyester (35 g/m²) |
| Wicking layer | Apertured viscose spunlace (50 g/m²) |
| 105 | Or |
| | Apertured bamboo spunlace (30 g/m²) |
| Absorbent | Pulp and SAP (300 g/m²) |
| structure 106 | |
| Garment- | Polyethylene film (20 μm thickness) |
| facing layer 107 | |
| Adhesive | 3M spray adhesive |

### Example 2—Materials

It may be of interest to exchange materials in the absorbent articles for compostable materials. Consequently, the inventors have investigated a variety of home and industrially compostable materials and exemplary materials for each component are provided in Table 2, below.

TABLE 2

| | Recommended | Recommended | |
|---|---|---|---|
| | material | material | Further home |
| | (home | (industrially | compostable |
| Component | compostable) | compostable) | materials |
| Topsheet | Hydrophobic | Spunbond or | PHA spunbond/ |
| | cotton or | spunlace | spunlace |
| | Tencel spunlace | polylactic | |
| | | acid (PLA) | |
| ADL | Pulp + Binder | Air-through | Air laid cotton |
| | | bonded PLA | ADL |
| Garment- | Thermoplastic | Biodegradable | Bio-based/Home |
| facing layer | Starch (TPS) | polyethylene | compostable PE |
| | | (PE) | |

| Absorbent | Pulp/Potassium polyacrylate powder | |
|---|---|---|
| structure | | |
| Wicking layer | Apertured spunlace viscose/bamboo | |
| Adhesive | Rosin-based or Terpene | Bio-based polymer |
| | based adhesives | Adhesives |

### Example 3—Liquid Wicking Rate (Capillarity)

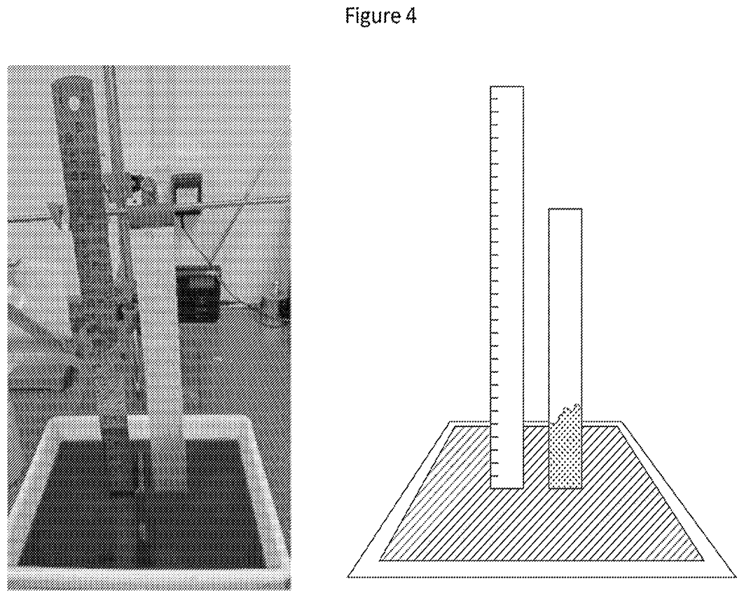

A liquid wicking rate assessment of different wicking materials was carried out according to EDANA WSP 101.1.RO (20) as is detailed above. Exemplary apparatus for performing this experiment is shown in FIG. 4.

Each specimen was placed vertically in 15 mm of fluid (0.9% saline solution) and liquid rise (wicking distance) was recorded at 10, 30, 60 and 300 seconds.

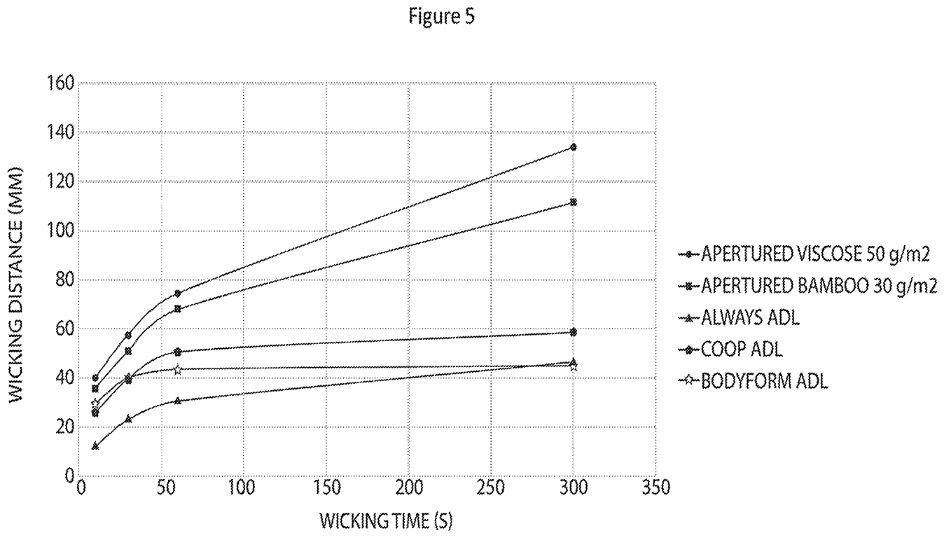

Test specimens as shown in the results of FIG. 5 were:
1. Spunlace apertured viscose 50 g/m2
2. Spunlace apertured bamboo 30 g/m2
3. Commercial ADL from an Always Ultra pad
4. Commercial ADL from a COOP branded pad
5. Commercial ADL from a Bodyform pad

Results of liquid wicking rate tests performed in accordance with the standard method are shown in FIG. 5. Apertured viscose and bamboo performed better or equivalently to the ADL layers of commercial sanitary towels in terms of liquid wicking rate.

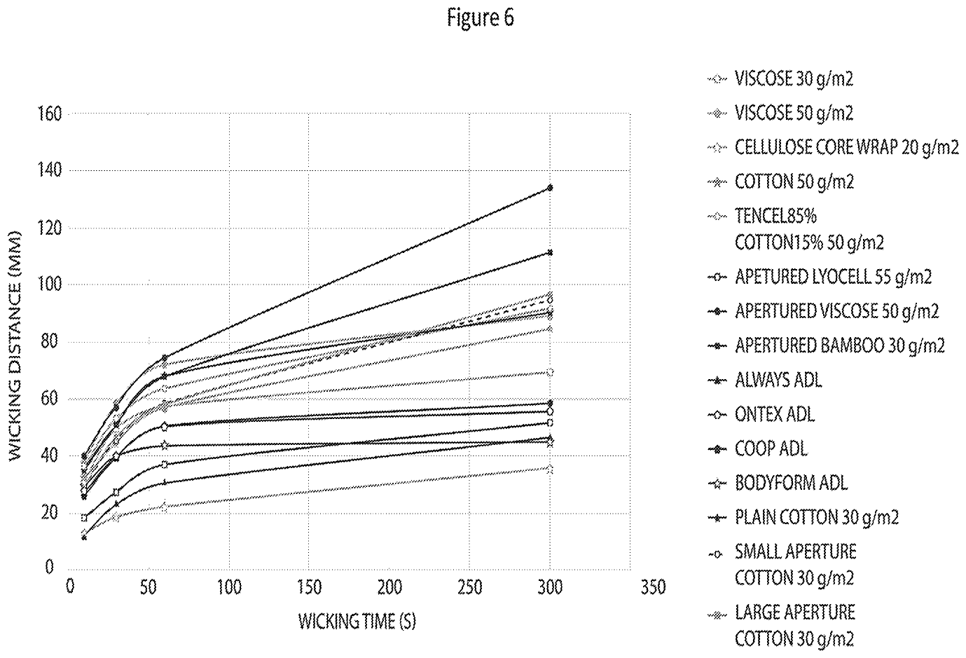

Further wicking materials were also tested for comparative purposes, using the same standard procedure. The results shown in FIG. 6 indicate that wicking layers prepared from a range of natural and man-made materials perform adequately in terms of wicking rate. The majority of wicking materials tested for use in the invention wicked fluid at a faster rate than the ADL layers of commercial sanitary towels (Always ADL, Ontex ADL, COOP ADL and Bodyform ADL).

### Example 4—Liquid Absorptive Capacity

Experiments were undertaken to examine the liquid absorptive capacity of wicking layers comprising spunlace apertured viscose and spunlace apertured bamboo. The tests were carried out according to EDANA NWSP 101.1.RO (20) (as detailed above).

Samples were submerged below the surface of a 0.9% saline solution for 60 seconds, then hung freely vertical to drain for 120 seconds. The liquid absorptive capacity was then calculated from dry and final wet weights.

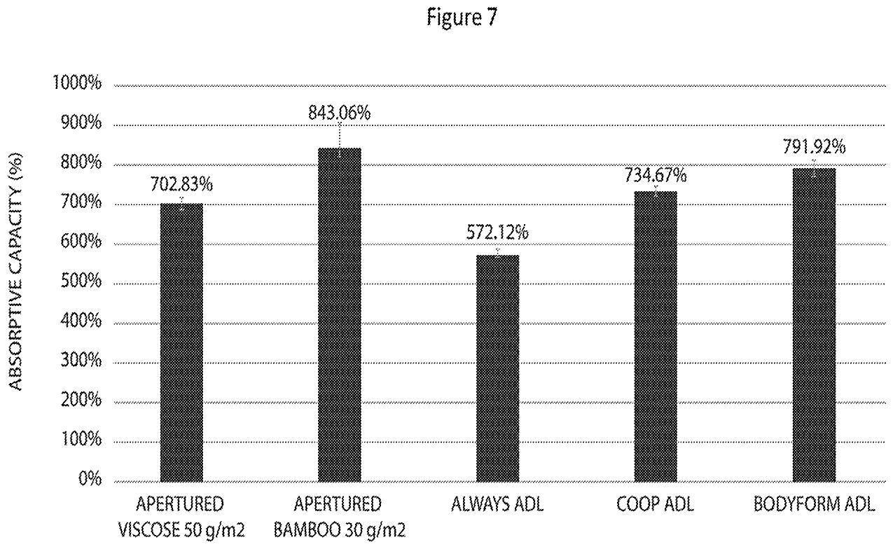

Test specimens as shown in results of FIG. 7 were:
1. Spunlace apertured viscose 50 g/m2
2. Spunlace apertured bamboo 30 g/m2
3. Commercial ADL from an Always Ultra pad
4. Commercial ADL from a COOP branded pad
5. Commercial ADL from a Bodyform pad

Results of liquid absorptive capacity tests performed in accordance with the standard method are shown in FIG. 7. Apertured viscose and bamboo performed better or equivalently to the ADL layers of commercial sanitary towels in terms of liquid absorptive capacity.

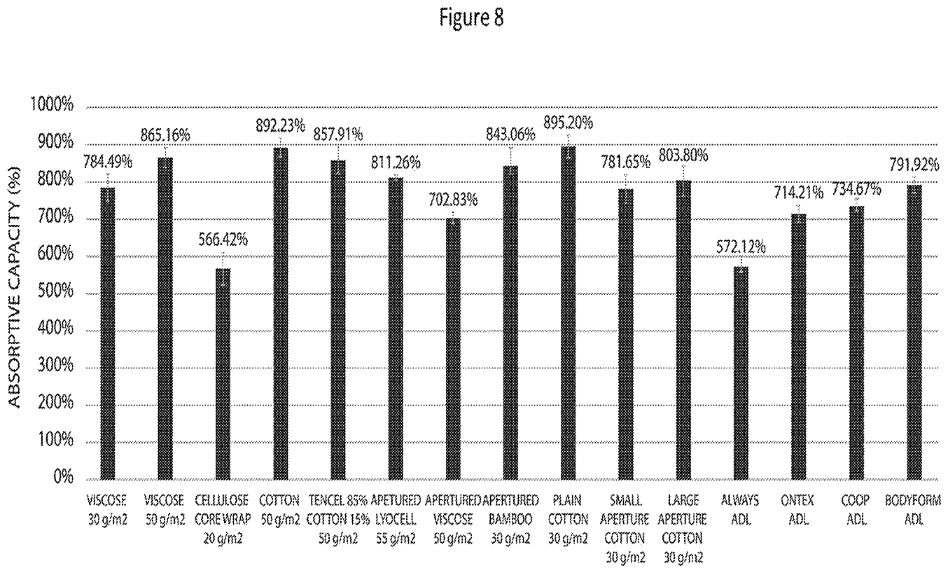

The absorptive capacity of further wicking materials was also tested for comparative purposes, using the same standard procedure. The results for these materials (shown in FIG. 8) indicate that wicking layers prepared from a range of natural and man-made materials perform adequately in terms of absorptive capacity. The majority of wicking materials tested for use in the invention had a higher liquid absorptive capacity than the ADL layers of commercial sanitary towels (Always ADL, Ontex ADL, COOP ADL and Bodyform ADL).

### Example 5—Liquid Spreading Area Assessment

Three sanitary napkins were compared in the liquid spreading area assessment, which were: a ‘Reference’ pad (no wicking layer); a ‘Bamboo’ pad, which was similar to the reference pad but contained a wicking layer of spunlace apertured bamboo 30 g/m2; and a ‘Natracare’ super pad which is a commercially available high absorbency pad.

For the spreading area assessment, the pads were laid flat and four successive 5 ml inlets of a saline solution (1% NaCl) containing a colouring agent were applied. Each 5 ml inlet was applied to the centre of the pad using a syringe with a 60 second wait between inlets. The spreading area was measured using Image Analysis software and calculated after each 60 second time period for each volume.

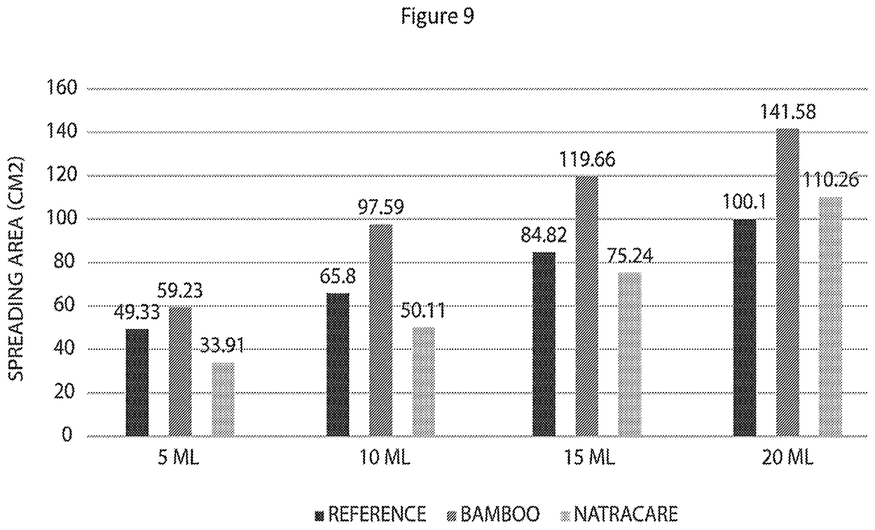

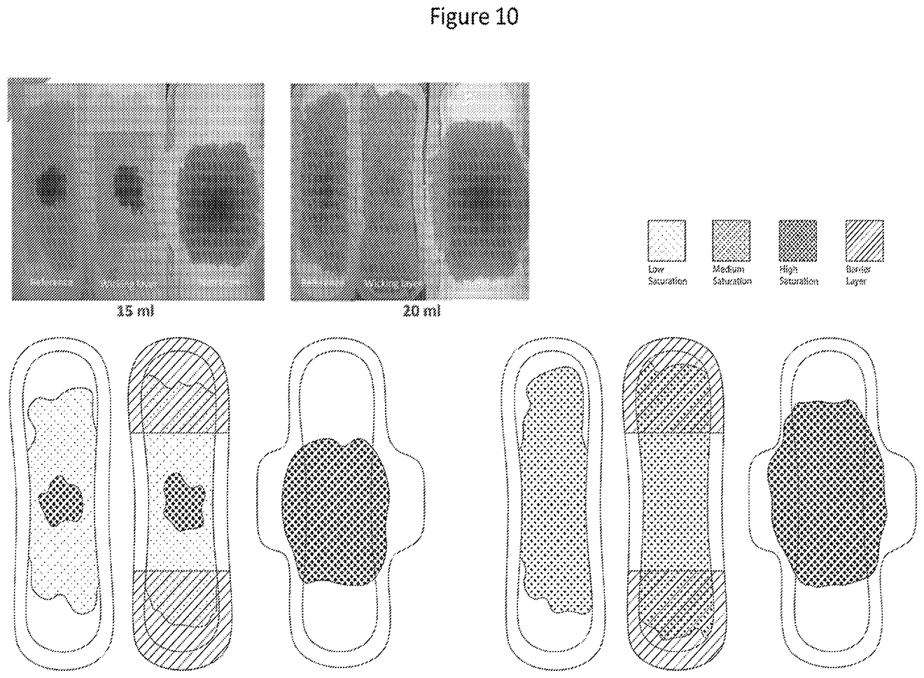

The results of the pad spreading assessment are shown in the graph of FIG. 9 and in the images of FIG. 10. As is seen from FIG. 9 the pad containing the bamboo wicking layer exhibited the greatest surface area spread of liquid at all time points measured.

FIG. 10 shows photographic comparisons of liquid spreading behaviour in each pad. Representative drawings of the photographs are also provided in this figure indicating the areas which have different levels of saturation (high, medium and low saturation). In the Natracare pad it can be seen that liquid is concentrated in one area of high saturation close to the point of ingress at the centre of the pad. The high saturation of liquid in this area presents a clear risk that the liquid will leak from the sides of the Natracare pad, as is evident in the photograph. By contrast, the pad containing the wicking layer and barrier sheet exhibited the highest surface area of medium and low liquid saturation, greatly reducing the risk of leakage from the pad.

### Example 6—Placement of the Barrier Sheet

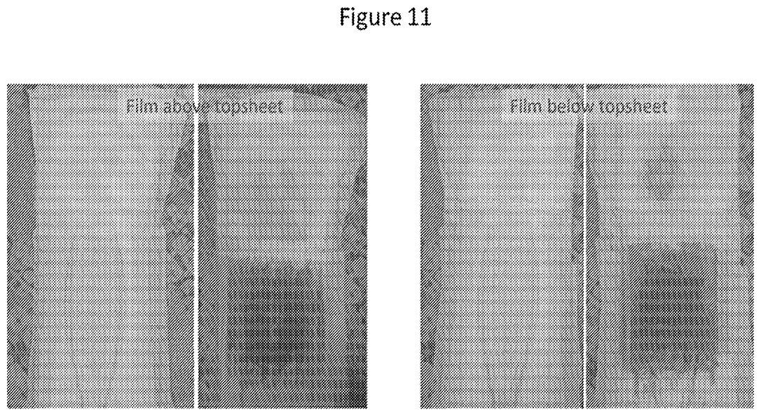

Two designs were trialled, in which a barrier sheet was placed either above or below a topsheet of a sanitary pad. The aim of the experiment was to assess whether placement of the barrier sheet would have an effect on moisture leakage from the end portions of a sanitary pad. The results are shown in FIG. 11. Neither design resulted in leakage of moisture from the areas covered with barrier sheet; therefore, it was concluded that either placement would adequately prevent rewetting from the end portions of the absorbent article.

### Example 6—Adhesion of Pad

The above embodiments are to be understood as illustrative examples of the invention. Further embodiments of the invention are envisaged. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

What is claimed is:

1. An absorbent article comprising a body-facing layer;
a liquid-impermeable garment-facing layer; and
an absorbent structure therebetween;
wherein the absorbent article further comprises
a wicking layer disposed between the body-facing layer and the absorbent structure; and
an acquisition/dispersion (ADL) layer disposed on the opposite side of the absorbent structure from the garment-facing layer;
wherein the body-facing layer comprises at least one barrier sheet, wherein the barrier sheet is substantially liquid-impermeable and is discontinuous, to allow the passage of liquid therethrough.

2. The absorbent article according to claim 1, wherein the wicking layer is arranged to facilitate a lateral movement of liquid from a central portion of the article towards one or more peripheral portions of the article.

3. The absorbent article according to claim 1, wherein the wicking layer is arranged to facilitate a lateral movement of liquid from a central portion of the article towards a front portion and a rear portion of the article.

4. The absorbent article according to claim 1, wherein the wicking layer comprises cellulosic fibres.

5. The absorbent article according to claim 1, wherein the wicking layer comprises parallel laid fibres, wherein the fibres are aligned with a longitudinal axis of the article.

6. The absorbent article according to claim 1, wherein a front portion and a rear portion of the body-facing layer comprise the barrier sheet, and a central portion of the body-facing layer is substantially free from the barrier sheet.

7. The absorbent article according to claim 1, wherein the barrier sheet is continuous over a front portion and a rear portion of the body-facing layer.

8. The absorbent article according to claim 1, wherein the barrier sheet is arranged to prevent rewetting at peripheral portions of the body-facing layer.

9. The absorbent article according to claim 1, wherein 30%-70% of the surface area of the body-facing layer is free from the barrier sheet.

10. The absorbent article according to claim 1, wherein at least 20% of the surface area of the body-facing layer comprises the barrier sheet, and wherein the end portions of the body-facing layer comprise substantially equal proportions of the barrier sheet.

11. The absorbent article according to claim 1, wherein the barrier sheet comprises a flexible plastic film.

12. The absorbent article according to claim 1, wherein the article is a sanitary napkin or an incontinence pad.
